(19) United States
(12) Patent Application Publication
(10) Pub. No.: US 2006/0004252 A1
(43) Pub. Date: Jan. 5, 2006

(54) Beaded bracelet for male genitalia

(76) Inventor: Tonya K. Astala, Grove City, PA (US)

Correspondence Address: LITMAN LAW OFFICES, LTD, PO BOX 15035, CRYSTAL CITY STATION, ARLINGTON, VA, 22215, US

(21) Appl. No.: 11/061,455

(22) Filed: Feb. 22, 2005

Related U.S. Application Data
(63) Continuation-in-part of application No. 29/208,015, filed on Jun. 24, 2004.

(51) Int. Cl. A44C 5/00; A61F 5/00

(52) U.S. Cl. 600/38; 63/11

(57) ABSTRACT

The beaded bracelet for male genitalia is a series of beads of any practicable size, shape, color, and/or configuration, strung upon a length of thin elastic thread which is secured to form a permanently closed loop. The relaxed diameter of the device is larger than the finger, with the diameter of the extended thread still being too small to fit about the hand or foot for use in the conventional locations on the body. The bracelet is configured for use as a reward for encouraging men to expose themselves in private and/or adult settings, and may be placed about the penis of a man who exposes himself as a reward for such an action. The bracelet may also be used to add a touch of levity to a relationship, lowering inhibitions and facilitating the enjoyment of sex between consenting adults.

### CROSS-REFERENCE TO RELATED APPLICATIONS

This is a continuation-in-part of my prior application Serial No. 29/208/015, filed Jun. 24, 2004.

### BACKGROUND OF THE INVENTION

1. Field of the Invention

The present invention relates generally to jewelry and other ornamentation for the human body. More specifically, the present invention comprises several embodiments of a beaded bracelet having a closed elastic loop and configured for fitting over the penis. The present device provides a number of different functions. In certain private and/or adult settings, the bracelet may be used to encourage or reward men to expose themselves, with bracelets being applied to their penises after exposure. The device may also provide both physical and psychological sexual stimulation as well.

2. Description of the Related Art

The concept of rewarding people for various activities is ornamental purposes, as indicators of rank and power, and for various other purposes, for centuries. Conventionally, such bracelets, necklaces and the like have been worn around the arm, wrist, or neck, or less frequently around the ankle.

More recently, with the promotion of the Mardi Gras celebration and revelry just before the beginning of Lent, large numbers of relatively inexpensive beads have been purchased by various groups and individuals to toss out to crowds during parades and other events. Similar events are held at various times and places throughout the U.S. In many instances, conventional rules are relaxed to a great extent, and spectators and participants alike are encouraged to abandon conventional standards, at least for the duration of the event. For example, it has become common for men to encourage women to expose their breasts during such event, with the reward for doing so generally being one or more strings of Mardi Gras beads or the like. However, this activity is generally performed at a distance between the men and women, as during a parade, with the beads being tossed from the men to the women; there is generally no direct physical contact between the men and women during this activity.

More recently, some women have felt that it is no more improper for them to initiate such an interaction, than for men to do so. Accordingly, some women have encouraged men, particularly physically attractive men, to expose themselves in return for receiving Mardi Gras beads or the like. However, at such events, there is generally no opportunity for direct physical contact between the men and women involved, with the reward beads being tossed from the women to the men for their exposure.

In certain parties and similar gatherings the opportunity may arise for closer interactions between participants, performers, and/or spectators. In fact, physical contact may be possible, and desired under certain circumstances, between people attending such an event. This makes it possible for a participant to bestow beads or other reward personally to or upon the person who has agreed to expose herself or himself. It has been conventional for a person or persons to place a string of Mardi Gras beads or the like about the neck of a person who has agreed to expose himself or herself at the request of the bead bestower(s), whether men or women, where close physical interaction is possible.

However, to the knowledge of the present inventor there has been no manufacture of a beaded bracelet or the like having a size configured to fit properly about the penis, to reward a man for exposing himself. The present inventor is aware of PCT Application No. 98/42,293 published on Oct. 1, 1998, titled “Sexual Stimulation Underwear,” which describes (according to the drawings and English abstract) an elongated beaded strand worn through the crotch of a woman and passing adjacent the vagina and clitoris. Japanese Patent No. 2001-231,799 published on Aug. 8, 2001 for a “Ring made of deodorizing and desweating rubber containing active carbon and sterilizing agent for male” (English title). The ring is adapted to fit around the recess of the glans of the penis, and act as an antibacteriological device to reduce bacteria and resultant odors produced thereby (according to the English abstract). Another Japanese Patent No. 2001-17,215 published on Jan. 23; 2001 describes (according to the drawings and English abstract) a protective cover for the tuft of fine fibers which may extend from a beaded bracelet or the like.

The present inventor is also aware of two websites (starlady.com/mg.htm and phdtop.com/smydla/) which discuss the Mardi Gras type activities noted further above, in which revelers are encouraged to expose themselves and have strings of beads tossed to them in reward.

None of the above inventions and patents, taken either singly or in combination, is seen to describe the instant invention as claimed. Thus a beaded bracelet for male genitalia solving the aforementioned problems is desired.

### SUMMARY OF THE INVENTION

The beaded bracelet for male genitalia is fabricated from a length of elastic material, e.g. a small diameter, woven or twisted elastic thread or cord capable of being extended up to about twice its relaxed length. A series of beads of various colors, shapes, sizes, and/or configurations is installed on the cord, with the beads perhaps having different geometric shapes, caricatures, and/or other features as desired. Some of the beads may be provided with phosphorescence or other means enabling them to glow in the dark or in dim light, if so desired.

The length of the thread is adjusted to provide a loop having a relaxed diameter of about an inch, with the extended thread loop diameter being on the order of two inches. The ends of the thread are then tied or otherwise secured to permanently close the loop. The present bracelet is thus too small to fit over the hand or foot for wear upon the wrist or ankle, but is too large to remain upon the finger if applied thereto. However, the diameter range provides a somewhat loose fit about the relaxed penis, in order to avoid discomfort, with the stretch of the elastic permitting the fingers of the hand to be inserted within the bracelet in order to facilitate application over the penis.

While the present bracelet is primarily intended as a person-to-person means of rewarding a certain act of behavior, i.e. a man exposing himself, it may provide other functions as well. The act of donning the present bracelet often results in some psychological (and therefore likely physical, as well) arousal for at least the man, and perhaps for both parties involved. It may also tend to lower any inhibitions between partners during sex, thereby facilitating the experience, and perhaps even making the experience possible for some couples who were unable to enjoy sex before.

These and other features of the present invention will become readily apparent upon further review of the following specification and drawings.

### BRIEF DESCRIPTION OF THE DRAWINGS

FIG. 1 is an environmental, perspective view of first embodiment of a beaded bracelet for male genitalia according to the present invention.

FIG. 2 is a perspective view of a second embodiment of the present bracelet, with the beads having a different configuration from the embodiment of FIG. 1.

FIG. 3 is a perspective view of a third embodiment of the present bracelet, in which multicolored beads are used.

FIG. 4 is a perspective view of a fourth embodiment of the present bracelet, in which beads of uniform color and configuration are used.

FIG. 5 is a perspective view of a fifth embodiment of the present bracelet, with some of the beads deleted and/or broken away to show the knotted, closed loop elastic cord.

Similar reference characters denote corresponding features consistently throughout the attached drawings.

### DETAILED DESCRIPTION OF THE PREFERRED EMBODIMENTS

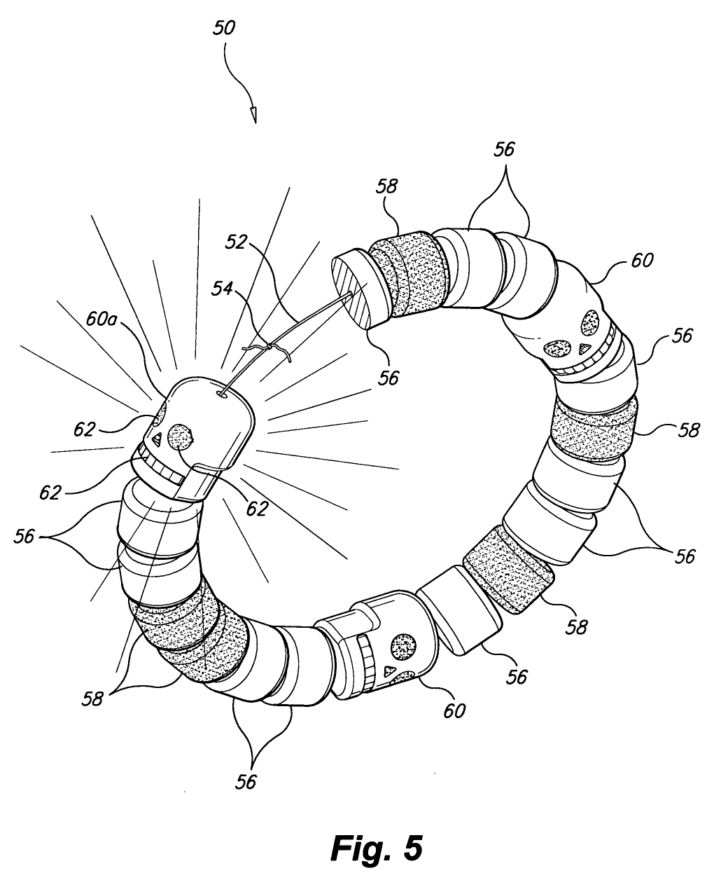

The present invention comprises a series of embodiments of a beaded bracelet for placement upon the male genitalia, more specifically for placement about the penis. The present bracelet may be used as a reward to encourage men to expose themselves in certain adult situations, and/or perhaps as a sexual stimulation device. FIG. 1 of the drawings provides a somewhat enlarged environmental perspective view (to show detail) of a first embodiment of the present bracelet 10, shown placed upon a penis P. The bracelet 10 comprises a length of very fine diameter twisted or woven elastic material having a series of beads or the like threaded thereon. The two ends of the elastic thread are tied or otherwise permanently secured together once the beads have been strung thereon, to form a permanently closed loop. Details of this assembly are shown more clearly in the embodiment of FIG. 5, and discussed in detail further below.

The loop of resilient or elastic thread of the present bracelet 10 is formed to have a relaxed diameter of about one inch, more or less, in order to fit comfortably about the adult penis. In any event, the loop should have a relaxed diameter somewhat larger than an adult human finger in order to preclude installation and use as a finger ring, and a fully extended diameter somewhat smaller than an adult human hand or foot, in order to preclude passage over the hand or foot for use as a wristlet or anklet. The resilient elastic thread used in the forming of the present bracelet embodiments has a maximally extended length of about twice its relaxed length.

The beads forming the bracelet 10 and other embodiments may be of any practicable size, shape, configuration, color, transparency, translucency, opacity, etc., as desired. Sufficient beads are placed upon the elastic thread so as to essentially completely cover and conceal the elastic thread running through each of the beads. The bracelet 10 of FIG. 1 comprises a series of circular beads 12 with a number of flattened, star-shaped beads 14 interspersed therewith. It should be noted that the holes or passages through the star-shaped beads 14 pass generally diametrically therethrough, in order to allow the beads 14 to be turned about the thread loop to orient the smooth flats 16 to face the inner and outer circumferences of the bracelet 10. In this manner, only a smooth surface contacts the relatively thin and sensitive skin of the penis, thereby precluding discomfort and irritation when placed thereon. This is not so critical for the flattened circular beads 12 comprising the remainder of the bead assembly, with these circular beads 12 having axially formed holes or passages with their rounded cylindrical circumferences forming the inner and outer peripheries of the bracelet 10.

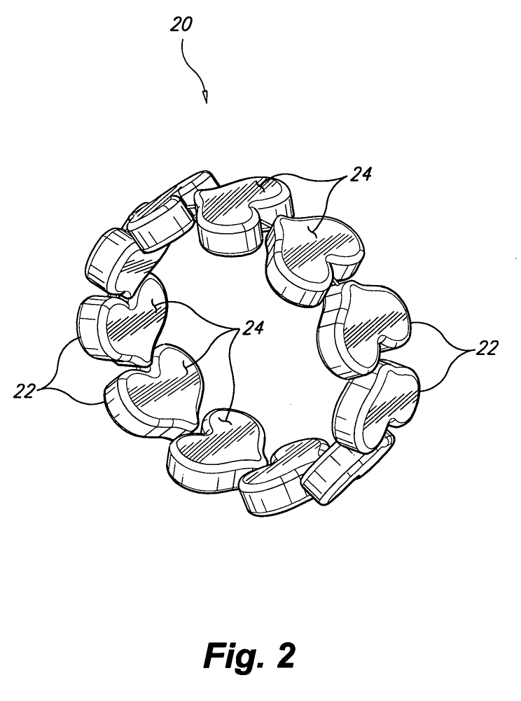

FIG. 2 of the drawings provides a somewhat enlarged illustration of a second embodiment bracelet 20. The bracelet 20 is formed in the same manner as the bracelet 10, but utilizes a series of flattened, heart shaped beads 22. As in the case of the star shaped beads 14 of the bracelet 10 of FIG. 1, the hole or passage for the thread loop is formed generally diametrically therethrough, i.e. parallel to the opposed parallel flat surfaces 24, for the same reason as the diametric orientation of the thread passages through the star shaped beads 14 of the bracelet 10 of FIG. 1. It will be seen that other bead configurations may be interspersed with the heart shaped beads 22 of the bracelet 20 as desired, as in the case of the two different bead configurations of the bracelet 10 of FIG. 1.

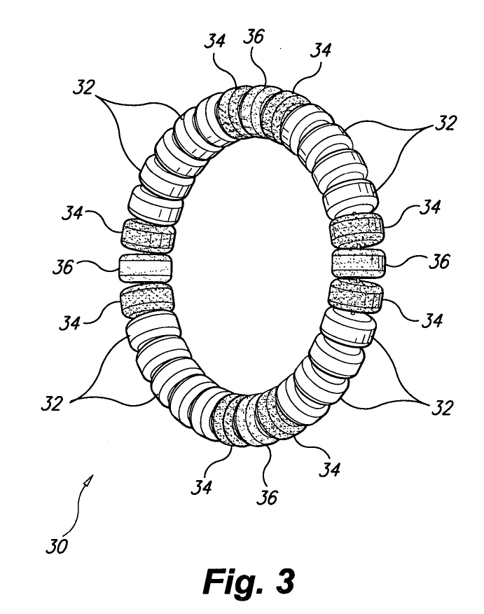

FIG. 3 provides an enlarged perspective illustration of yet another embodiment of the present bracelet, designated as bracelet 30. The bracelet 30 is formed using essentially the same resilient thread loop structure as the other embodiments, but incorporates a series of beads 32, 34, and 36 having different colors from one another. While the beads 32 through 36 all have a flat, cylindrical configuration similar to the beads 12 of the bracelet 10 of FIG. 1, it will be seen that other bead shapes may be used as desired, e.g. the star shaped beads 14 of the bracelet 10 of FIG. 1 and/or the heart shaped beads 22 of the bracelet 20 of FIG. 2, and/or other shapes and configurations as desired, in any color, shade, tint, or degree of transparency or opacity desired.

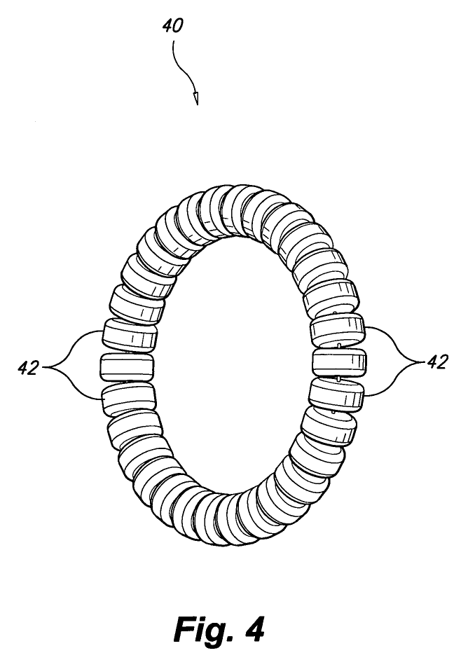

FIG. 4 is an enlarged perspective view of still another embodiment of the present invention, comprising a beaded bracelet 40 having a string of identically colored and configured beads 42 thereon. The beads 42 are essentially the same color and configuration as the flattened cylindrical beads 12 of the bracelet 10 of FIG. 1 and the beads 32 of the bracelet 30 of FIG. 3. As in the case of the other bracelet embodiments, beads having different configurations than those illustrated in FIG. 3, and/or different from one another, may be used in lieu of the uniform bead 32 configuration of the bracelet 30 of FIG. 4.

FIG. 5 provides an enlarged perspective view of a further embodiment 50 of the present bracelet, with some of the beads deleted and/or partially broken away in order to illustrate the resilient elastic thread loop which passes through the beads. The thread loop 52 is formed of a length of very fine diameter elastic or resilient thread, which may be twisted or woven of a number of smaller, very fine elastic fibers or strands. The relaxed diameter, i.e. measured across the width of the thread 52, of the material is preferably no greater than about one millimeter. This thread gauge or diameter provides sufficient strength for the purpose of the device, while allowing the thread 52 to be stretched to its maximum bracelet diameter with minimal force being required.

The bracelet 50 (and other embodiments as well) is formed by cutting an appropriate length of elastic thread material, e.g. five inches or so, and stringing a series of beads thereon as desired. A total length of about three inches of beads are strung sequentially along the elastic thread 52, with the beads being immediately adjacent and in contact with one another. The elastic thread 52 is then stretched slightly from the beads and the free ends of the thread 52 are then permanently secured together, e.g. by tying a knot 54 or other means as desired. The loose ends of the knot 54 are clipped as required, and when the thread 52 is relaxed, the beads are pulled together to form an essentially unbroken, continuous length or string of beads about the closed thread loop 52.

In addition to the details of the thread loop 52 used in forming the various bracelet embodiments, FIG. 5 also illustrates a series of different bead configurations. The bracelet 50 of FIG. 5 includes a series of somewhat short, cylindrical beads 56 and 58, with the beads 56 and 58 being differentiated by their different colors. In addition to the beads 56 and 58, one or more caricature beads 60 are installed in the bracelet 50. These caricature beads 60 may be representations of human skulls, as depicted in FIG. 5, and/or may be any other caricature as desired. As in the cases of the other bracelets 10 through 40 of FIGS. 1 through 4, the various beads 44 through 58 of the bracelet 50 are preferably smoothly contoured in order to avoid irritation or discomfort to a man wearing the bracelet.

Any of the various beads used in forming any of the present bracelets may be of any color, shading, tint, transparency, opacity, or other characteristic, as desired. In addition to these properties, the beads may be formed of a material which is capable of phosphorescence for a period of time after being exposed to light. One of the skull caricature beads 60a of FIG. 5 is shown as emitting such phosphorescence. Certain plastics are capable of producing phosphorescence for a period of time after exposure to light, and any of the various bead configurations which may be used in the present bracelet embodiments may be formed of such plastic, or other material(s) as desired. Where caricature beads are used, it may be desired to provide such phosphorescence only in areas 62 representing the eyes, nose, mouth, and/or ears, etc. of the facial area. Variations on this theme may be developed as desired.

The present beaded bracelet in its various embodiments may be placed about the penis of any consenting male. Such a situation or opportunity may present itself at any number of private and/or adult settings where bodily exposure is expected, permitted, or performed by custom. An example is the Mardi Gras celebration in New Orleans, where it has been known for years for consenting women to expose their breasts under sufficient encouragement. The reward for doing so is generally one or more strings of Mardi Gras beads. More recently, many women (as well as some homosexual men) have concluded that equality allows them to request men to expose themselves as well, with equivalent rewards (i.e., a string of Mardi Gras beads) being tossed to men willing to accept the challenge.

In many instances, the physical proximity of the challenger or requester and the man exposing himself is sufficiently close to permit contact between the two. Accordingly, the present invention provides an alternative means for encouraging a man to expose himself. A woman (or another man) wishing to see a man expose himself, may provide verbal encouragement for such an act with the promise of the bestowal of a bracelet of the present invention upon the man in return for his exposure. The man exposing himself in response to the challenge or request is rewarded by having the challenging or requesting person place one (or more) of the present bracelet(s) upon the exposed penis of the man.

In some instances, such encouragement may not be sufficient, but it may be possible to encourage such activity by means of a sufficiently interesting bracelet. Accordingly, the present bracelets may be formed to include beads having various characteristics relating to profession, hobby, or other interest of the male. This is particularly true where gatherings occur with participants having a common interest, e.g. a motorcycle rally or the like. The addition of phosphorescent properties to one or more of the beads may provide additional interest, particularly in low light environments.

The present bracelet in one or more of its various embodiments may also serve as a sexual stimulation device. In many instances, it may be difficult for a man to become physically aroused. Oftentimes this may be due to psychological problems, such as workplace or other distractions which have been carried over, or other reasons. In many instances, sex has become routine to the couple engaging in the activity, and some new variation may enable arousal or add to the pleasure of the activity. Accordingly, one (or more) of the present bracelet(s) may be placed upon the penis, with the attention given and the physical sensation adding to the psychological and physical arousal desired.

In conclusion, the present beaded bracelet in its numerous embodiments provides an intriguing means for women (or even some men) to return the favor wherein men have traditionally asked women to expose themselves, with the women being offered some form of small reward. The present invention allows women to reverse the roles, and moreover provides a reward involving close and personal physical contact. The present bracelets in their various forms will thus provide a novel and entertaining means of encouraging male exposure for consenting adults in adult situations and environments where such activity is permitted.

It is to be understood that the present invention is not limited to the embodiments described above, but encompasses any and all embodiments within the scope of the following claims.

I claim:

1. A beaded bracelet for male genitalia, comprising:
a permanently closed loop of elastic thread, the loop having a relaxed diameter and a fully extended diameter; and
a plurality of beads strung along the thread covering and concealing the entire thread.

2. The beaded bracelet for male genitalia according to claim 1, wherein:
the thread is twisted from a plurality of fine elastic fibers; and
the thread has a relaxed diameter no greater than about one millimeter.

3. The beaded bracelet for male genitalia according to claim 1, wherein the relaxed diameter of said loop is about one inch.

4. The beaded bracelet for male genitalia according to claim 1, wherein said beads have inwardly oriented smooth surfaces for precluding discomfort and irritation when placed upon the penis.

5. The beaded bracelet for male genitalia according to claim 1, wherein said beads comprise a mixture of different shapes.

6. The beaded bracelet for male genitalia according to claim 1, wherein said beads comprise a mixture of different colors.

7. The beaded bracelet for male genitalia according to claim 1, wherein at least one of said beads is a caricature.

8. The beaded bracelet for male genitalia according to claim 1, wherein at least one of said beads is at least temporarily phosphorescent to glow in the dark.

9. A method of rewarding exhibitionist behavior, comprising the steps of:
(a) encouraging at least one male to expose his genitalia; and
(b) bestowing a beaded bracelet upon the exposed penis of the at least one male exposing himself as a result of the encouragement.

10. The method of rewarding exhibitionist behavior according to claim 9, wherein at least some beads upon the bracelet have interesting characteristics to the at least one male.

11. The method of rewarding exhibitionist behavior according to claim 9, wherein the beads have at least one smooth surface, the method further including the step of orienting the at least one smooth surface of each of the beads inwardly about the bracelet, thereby precluding discomfort and irritation when placed upon the penis.

12. The method of rewarding exhibitionist behavior according to the method of claim 9, wherein at least one of the beads is at least temporarily phosphorescent to glow in the dark.
